(19) United States
(12) Patent Application Publication
(10) Pub. No.: US 2011/0060417 A1
(43) Pub. Date: Mar. 10, 2011

(54) SHOULDER JOINT PROSTHESIS, ESPECIALLY REVERSE SHOULDER JOINT PROSTHESIS

(75) Inventors: Beat R. Simmen, Tagelswangen (CH); Marc A. Riner, Aristau (CH); Ernst Kehrli, Rotkreuz (CH)

(73) Assignee: SMITH & NEPHEW ORTHOPAEDICS AG, Rotkreuz (CH)

(21) Appl. No.: 12/918,620

(22) PCT Filed: Feb. 3, 2009
(86) PCT No.: PCT/EP09/00701

§ 371 Date: Nov. 18, 2010

(30) Foreign Application Priority Data
Feb. 21, 2008 (DE) 10 2008 010 478.7

(51) Int. Cl. A61F 2/40

(52) U.S. Cl. 623/19.11

(57) ABSTRACT

Shoulder joint prosthesis, especially a reverse shoulder joint prosthesis, having a joint part, especially a humeral socket (10), connectible at the proximal end of a humeral shaft (18). The joint part associated with the humeral shaft (18) is held displaceably relative to the shaft (18) at least in the antero-posterior direction.

The present invention relates to a shoulder joint prosthesis, especially a reverse shoulder joint prosthesis, in accordance with the preamble of claim 1.

Various diseases of the shoulder joint can necessitate the fitting of an artificial shoulder joint. Besides the visible joint changes on radiographic or magnetic resonance imaging, the indications for such an intervention are the patient's symptoms, amongst which there should primarily be mentioned the severe pain at rest and on loading, making it necessary for medication to be taken.

Alongside artificial hip and knee joint replacements, shoulder joint endoprosthetics have made great advances in recent years. In cases of painful destruction of the shoulder joint as a result of arthrosis (joint wear), rheumatoid arthritis (rheumatic inflammation of the joint), humeral head necrosis (dying-off of the head of the humerus) or fracture of the humeral head, replacement of the joint surfaces of the humeral head and of the shoulder socket using prostheses made from titanium and polyethylene is now possible with a high level of success.

In a not inconsiderable proportion of patients suffering from untreated massive rotator cuff tear (at least two torn rotator cuff tendons), arthrosis of the shoulder joint develops. In the case of massive rotator cuff tear, the humeral head migrates in an antero-superior direction and accordingly loses its “centring” in the socket of the joint. In addition, there is shortening and weakening of the deltoid muscle, which in the case of massive rotator cuff tear is still capable of raising the arm laterally. In this case, it is possible for the joint to be replaced using a so-called reverse prosthesis system (Grammont reverse Delta III prosthesis), in which a “hemisphere”, or glenosphere, is screwed onto the original socket and a concavely shaped “humeral socket” is fixed in the bone of the humerus. As a result, the upper arm is displaced back “down” and the function of the deltoid muscle improved.

At this point the considerations of the present invention come into play: The humeral head has been displaced relative to the axis of the upper arm. The consequence of this is that, in the case of a reverse shoulder joint prosthesis, the proximal portion is displaced too far in the anterior direction. This results, on the one hand, in a relatively large amount of bone being lost on the anterior side of the proximal humerus and, on the other hand, in a non-anatomic position of the metaphyseal portion of the implant.

In provision of the primary treatment, where the humeral head and glenoid are not exchanged, the afore-mentioned problem is solved by eccentrically arranging the joint heads at the proximal end of the humerus in order to obtain an anatomically correct position of the joint head.

In the case of reverse prostheses, where a glenosphere is mounted on a scapular baseplate, there are no known appropriate solutions for avoiding the afore-mentioned problems.

The problem underlying the present invention is accordingly a shoulder joint prosthesis, especially a reverse shoulder joint prosthesis, wherein the joint part associated with the humeral shaft can be adjusted in a manner appropriate to the anatomy, and which results in reduced loss of material (bone).

In accordance with the invention, the problem is solved by the characterising features of claim 1.

A fundamental aspect of the present invention is accordingly the possibility of displacing that joint part of a shoulder joint prosthesis which is associated with the humeral shaft at least in the antero-posterior direction. As a result, this part can be better matched to the anatomy of the patient. This applies especially to reverse shoulder joint prostheses having a humeral socket connected to the humeral shaft.

Advantageous embodiments and structural details of the present invention are described in the subordinate claims. In this context it should be pointed out that the approach according to the invention is suitable not only for reverse shoulder joint prostheses but also for customary shoulder joint prostheses for provision of the primary treatment.

Very often it is also necessary for that joint part of the shoulder joint prosthesis which is associated with the humeral shaft to be displaced upwards or downwards in order to allow the joint to be positioned in a manner appropriate to the anatomy. This is possible, for example, by means of the fact that between the joint part on the shaft side, for example the humeral socket, and the shaft two eccentricities are arranged and superimposed. As a result, the joint part associated with the shaft, for example the humeral socket in the case of the reverse arrangement, is displaceable both in the antero-posterior direction and also in a direction approximately perpendicular thereto, or the medio-lateral direction.

Fixing of the parts that are displaceable relative to one another is accomplished by means of a fastening and securing screw of a customary kind.

That joint part of the shoulder joint prosthesis which is associated with the humeral shaft, in the case of the reverse arrangement thereof the humeral socket, is displaceable relative to the shaft or to an adaptor arranged between the shaft and the joint part, or humeral socket, either steplessly or stepwise.

Insofar as displacement only in the antero-posterior direction is to be possible, the joint part on the shaft side and the shaft, or an adaptor arranged between them, include complementary guiding means, especially longitudinal guiding means, on their sides facing one another.

In order to facilitate indexed displacement of the joint part associated with the shaft, especially intra-operatively, this joint part or the shaft or an adaptor arranged between them has, on the side thereof facing the respective other part of the prosthesis, and in the implanted state extending in the antero-posterior direction, a row of teeth which co-operates with at least one tooth on the respective other part of the prosthesis for stepwise displacement, in the antero-posterior direction, of the joint part associated with the shaft. The step size is preferably about 0.2 mm to 0.8 mm, especially about 0.5 mm. Preference is given to the one tooth on the respective other part of the prosthesis also being replaced by a row of teeth extending in the antero-posterior direction. As a result, on stepwise displacement, relative to the shaft, of the joint part on the shaft side, even more secure holding of the particular adjusted relative position is obtained.

It can moreover be advantageous for the joint part associated with the humeral shaft, in the case of a reverse shoulder joint prosthesis the humeral socket, to be held displaceably or displaced relative to the shaft also in the proximal-distal direction.

In order to make available all conceivable degrees of freedom, the joint part associated with the humeral shaft, in the case of a reverse shoulder joint prosthesis the humeral socket, can also be held so as to be tiltable relative to the shaft, at least in the antero-posterior direction, preferably in the manner of a universal joint.

It should also be mentioned at this point that the joint part on the humerus side, in the case of a reverse shoulder joint prosthesis the humeral socket, is preferably of two-part construction, that is to say it comprises a bearing part defining a joint surface made of polyethylene, ceramics, stainless steel or like bearing material tolerated by humans and a bearing mounting part by way of which the connection to the humeral shaft is made. Preference is given to the bearing part, which defines a convex or, in the case of a reverse shoulder joint prosthesis, a concave bearing surface, being able to be latched into place in the bearing mounting part. Latching is carried out in such a way that, if necessary, revision of the bearing part is also possible without the implant otherwise having to be modified.

As an alternative to the above-described arrangement, in order to solve the set problem, it is also feasible to provide a set of joint parts, in the case of a reverse shoulder joint prosthesis a set of humeral sockets, the joint surfaces of which, especially the geometric mid-axes thereof relative to the shaft or to the geometric longitudinal mid-axis of the latter, are arranged or constructed so as to be displaced to differing extents in the postero-anterior direction and/or, if necessary, in the medio-lateral direction and/or, if necessary, also in the proximal-distal direction. Preference is given to the set of joint parts comprising a total of five joint parts, wherein one of the joint parts defines a joint surface whose geometric mid-axis is located in the plane of the geometric longitudinal mid-axis of the shaft. The geometric mid-axes of the joint surfaces of the other joint parts are displaced relative to the geometric longitudinal mid-axis of the shaft to differing extents in the posterior and anterior direction etc.

The maximum displacement in the postero-anterior direction and/or in the medio-lateral direction and/or in the proximal-distal direction is preferably in the range between 3.0 mm to 10.0 mm, especially approximately in the range between 4.0 mm and 8.0 mm. In this regard, a plurality of sets of joint parts, for example humeral sockets, can be provided which in each case differ in terms of the maximum displacement in the postero-anterior direction etc.

The step size between the individual displacement steps is in the range from about 0.5 mm to 2.0 mm, especially about 1.0 mm to 1.5 mm.

It is also feasible to provide a set of joint parts, in the case of a reverse shoulder joint prosthesis a set of humeral sockets, the joint surfaces of which have angles of inclination of differing magnitudes relative to the shaft or to the longitudinal mid-axis of the latter. In the case of this embodiment too, all degrees of freedom should therefore, as far as possible, be made available to the surgeon.

Arrangements of the humerus-side part of a reverse shoulder joint prosthesis constructed in accordance with the invention will be explained hereinbelow in further detail with reference to the accompanying drawings, in which:

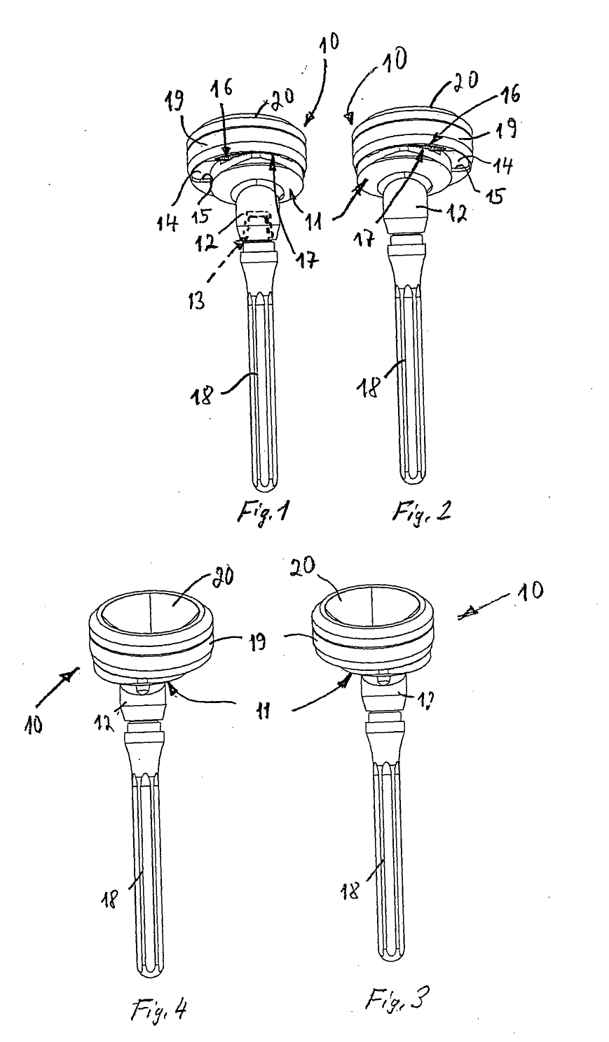

FIG. 1 shows that part of a reverse shoulder joint prosthesis which is associated with the humerus, in a view from the lateral direction, the humeral socket being located in the posterior or the anterior end position;

FIG. 2 is a view corresponding to FIG. 1, the humeral socket being located in the opposite end position to FIG. 1;

FIGS. 3 and 4 show that part of the reverse shoulder joint prosthesis which is associated with the humerus, corresponding to FIGS. 1 and 2 but in each case from the medial direction;

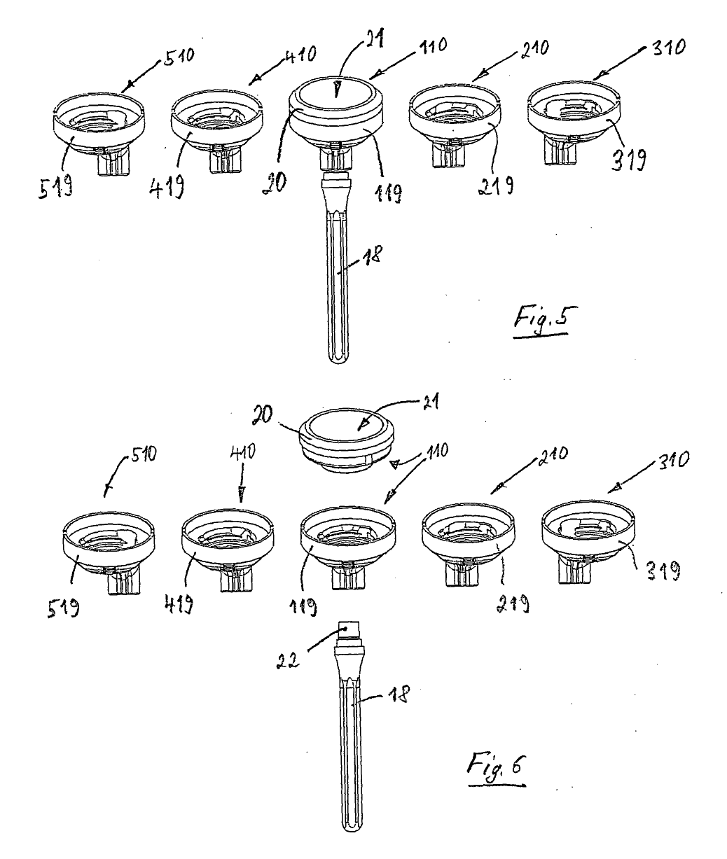

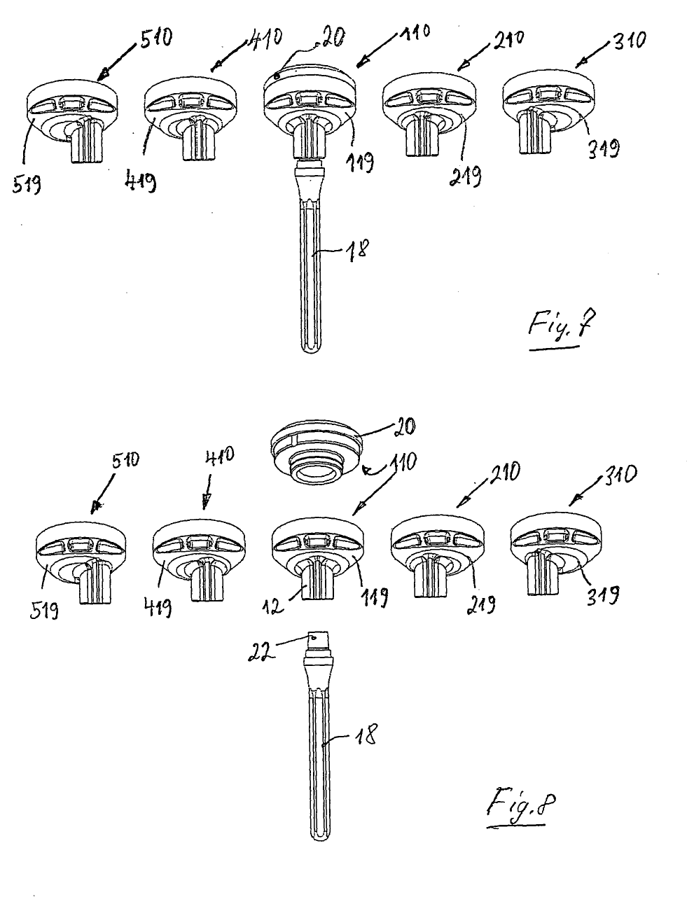

FIGS. 5-8 show a humeral shaft with which there is associated a set of, in each case, five humeral sockets, which define a joint surface that is displaced relative to the shaft to different extents anteriorly and posteriorly, more specifically in an assembled view from the medial direction (FIG. 5), in an exploded view from the medial direction (FIG. 6), in an assembled view from the lateral direction (FIG. 7) and in an exploded view from the lateral direction (FIG. 8).

That part of a reverse shoulder joint prosthesis which is associated with the humerus, and which is shown in FIGS. 1 to 4, includes a humeral socket 10, which includes a bearing insert 20 made preferably of polyethylene (PE) or ceramics and a bearing mounting part 19 made of material that is tolerated by humans, for example titanium or titanium alloy. This structural unit 10 is connected to the proximal end of a humeral shaft 19, more specifically in the case of the shown embodiment having a shaft connection member, or adaptor 11, located inbetween. The adaptor 11 is connected to the proximal end of the shaft 18 by means of a cone connection 13 known per se.

The humeral socket, or its bearing insert 20, co-operates in the implanted state with a glenosphere mounted on a scapular baseplate, more specifically thereby forming a complete reverse shoulder joint prosthesis.

As FIGS. 1 and 2, and likewise 3 and 4, show very well, the humeral socket 10 is held so as to be displaceable relative to the shaft 18, and more specifically in the case of the specific embodiment, relative to the adaptor 11, in a predetermined direction, more specifically in the antero-posterior direction. In the case of the shown embodiment, the displacement of the humeral shaft 10 is carried out stepwise. Alternatively, stepless displacement is also possible.

The humeral socket 10, or its bearing mounting part 19, on the one hand, and the adaptor 11, on the other hand, have complementary guiding means on their sides facing one another. Specifically, for this purpose, the humeral socket 10, or its bearing mounting part 19, has, on its side facing the adaptor 11, a recess 14 having a boundary wall 15 extending in the antero-posterior direction, which co-operates in longitudinally guiding manner with a complementary projection (not shown in further detail) on that side of the adaptor 11 which faces the humeral socket 10. A correspondingly reverse arrangement is of course also feasible.

Furthermore, FIGS. 1 and 2 especially show that both the humeral socket, or its bearing mounting part 19, and also the adaptor 11 each have on their sides facing one another a row of teeth 16, 17 which extends in the displacement direction, that is to say in this case in the antero-posterior direction, which co-operate for the purpose of stepwise displacement of the humeral socket 10 relative to the shaft 18 or relative to the adaptor 11 intermediately positioned between the humeral socket 10 and the shaft 18 in the direction predetermined by the longitudinal guiding means.

The arrangement shown is of modular construction. The humeral socket 10 can be adjusted intra-operatively.

It should furthermore be mentioned in respect of the arrangement shown that on that side of the adaptor 11 which faces the shaft 18 there is formed an extension 12 having an internal cone. This internal cone co-operates with an external cone formed on the proximal end of the shaft 18, more specifically thereby forming the afore-mentioned cone connection 13.

The relative position between the humeral socket 10 and adaptor 11 is secured by means of a fixing screw (not shown here in further detail).

As a result of the described arrangement it is no longer necessary to remove additional bone material anteriorly at the proximal end of the humerus in order to position the humeral socket in a manner appropriate to the anatomy. Furthermore, as a result of the described arrangement, the joint can be adjusted in a manner appropriate to the anatomy and in a manner customised to the patient. This applies especially to an arrangement in which the humeral socket is displaceable not only in the antero-posterior direction but also in a direction approximately perpendicular thereto, that is to say in the medio-lateral direction.

FIGS. 5 to 8 show various views (from the medial direction in FIGS. 5 and 6, and from the lateral direction in FIGS. 7 and 8), on the one hand in assembled form (see FIGS. 5 and 7) and on the other hand in exploded form (see FIGS. 6 and 8), of a set of humeral sockets 110, 210, 310, 410 and 510, the joint surfaces 21 of which, especially the geometric mid-axes thereof relative to the shaft 18 or to the geometric longitudinal mid-axis of the latter, are arranged or constructed so as to be displaced to differing extents in the postero-anterior direction. The humeral sockets 310 and 510 define the greatest displacement, the humeral sockets 210 and 410 less displacement, and the humeral socket 110 no displacement.

The maximum displacement in the postero-anterior direction should be in the range between 3.0 mm to 10.0 mm, especially approximately in the range between 4.0 mm and 8.0 mm. It is also feasible to provide a plurality of sets of humeral sockets which are distinguished by different maximum displacements in the postero-anterior direction and/or in the medio-lateral direction and/or in the proximal-distal direction. This is also dependent on the size of the individual patient (adult or child).

The step size between the individual displacement steps is in the range from about 0.5 mm to 2.0 mm, especially in the region of about 1.5 mm. This too is ultimately dependent on the circumstances governed by the patient.

It is otherwise also feasible to provide a set of joint parts, in the case of a reverse shoulder joint prosthesis a set of humeral sockets, the joint surfaces 21 of which have angles of inclination of differing magnitudes relative to the shaft 18 or to the longitudinal mid-axis of the latter. This embodiment can also be combined with an embodiment of the kind depicted here.

In this case too, the humeral sockets 110 to 510 are in each case of two-part construction. They comprise, on the one hand, a bearing insert or inlay 20 made of polyethylene, ceramics, stainless steel or like bearing material tolerated by humans and, on the other hand, a bearing mounting part 119, 219, 319, 419, 519. The inlay 20 is preferably able to be latched into place in the bearing mounting part 119 ff. so that, if necessary, it can also be replaced by another bearing part, especially a new bearing part, in the course of a revision.

The connection between the joint part, or humeral socket, and the shaft 18 is made by means of a cone connection corresponding to that shown in diagrammatic form in FIG. 1. For this purpose, the shaft 18 has on its proximal end a cone 22, which can be positioned inside an internal cone (not shown here in greater detail) in a lower extension 12 of the bearing mounting parts 119 to 519 in a manner known per se.

All features disclosed in the application documents are claimed as being important to the invention insofar as they are novel on their own or in combination compared with the prior art.

### REFERENCE NUMERALS

10 humeral socket
110 humeral socket
210 humeral socket
310 humeral socket
410 humeral socket
510 humeral socket
11 adaptor, or shaft connection member
12 central extension having an internal cone
13 cone connection between shaft and adaptor
14 recess
15 boundary wall of recess
16 row of teeth
17 row of teeth
18 humeral shaft
19 bearing mounting part
119 bearing mounting part
219 bearing mounting part
319 bearing mounting part
419 bearing mounting part
519 bearing mounting part
20 bearing insert, preferably made of PE or ceramics or like joint surface material
21 joint surface
22 cone

What is claimed is:

1. Shoulder joint prosthesis having a joint part, connectible at the proximal end of a humeral shaft, wherein the joint part associated with the humeral shaft, in the case of a reverse shoulder joint prosthesis the humeral socket, is held displaceably or displaced relative to the shaft at least in the antero-posterior direction.

2. Shoulder joint prosthesis according to claim 1, wherein the joint part associated with the humeral shaft, for example the humeral socket, is displaceable steplessly or stepwise.

3. Shoulder joint prosthesis according to claim 1, wherein the humerus-side part of the shoulder joint prosthesis is of modular construction, comprising
a joint part,
an adaptor and
a shaft.

4. Shoulder joint prosthesis according to claim 1, wherein the joint part associated with the humeral shaft, on the one hand, and the shaft or an adaptor arranged between the latter and the joint part, for example the humeral socket, on the other hand, have complementary guiding means, especially longitudinal guiding means, on their sides facing one another.

5. Shoulder joint prosthesis according to claim 4, wherein the longitudinal guidance means comprise a recess formed in the joint part, having at least one boundary wall extending in the antero-posterior direction and a complementary projection formed on the associated counterpart piece, or vice versa.

6. Shoulder joint prosthesis according to claim 1, wherein the joint part associated with the shaft, or the shaft or an adaptor arranged between the shaft and the joint part, has, on the side facing the respective other part of the prosthesis, and extending in the antero-posterior direction, a row of teeth which co-operates with at least one tooth on the respective other part of the prosthesis for stepwise displacement, in the antero-posterior direction, of the joint part associated with the humerus.

7. Shoulder joint prosthesis according to claim 1, wherein the joint part on the humerus side, by means of at least one screw, fixable and held in a predetermined relative position on the shaft or an adaptor arranged between the latter and the joint part.

8. Shoulder joint prosthesis according to claim 1, wherein the joint part on the humerus side, is held displaceably or displaced relative to the shaft also in a direction approximately perpendicular to the antero-posterior direction.

9. Shoulder joint prosthesis according to claim 1, between the joint part on the humerus side, and/or an intermediately positioned adaptor two eccentricities are superimposed so that the joint part, is displaceable both in the antero-posterior direction and also in a direction approximately perpendicular thereto, or the medio-lateral direction.

10. Shoulder joint prosthesis according to claim 1, wherein the joint part associated with the humeral shaft, in the case of a reverse shoulder joint prosthesis the humeral socket, is held displaceably or displaced relative to the shaft in the proximal-distal direction.

11. Shoulder joint prosthesis, especially according to claim 1, wherein there is provided a set of joint parts, in the case of a reverse shoulder joint prosthesis a set of humeral sockets, the joint surfaces of which, especially the geometric mid-axes thereof relative to the shaft or to the geometric longitudinal mid-axis of the latter, are arranged or constructed so as to be displaced to differing extents in the postero-anterior direction and/or in the medio-lateral direction and/or in the proximal-distal direction.

12. Shoulder joint prosthesis according to claim 11, wherein the maximum displacement in the postero-anterior direction and/or in the medio lateral direction and/or in the proximal-distal direction is in the range between 3.0 mm to 10.0 mm.

13. Shoulder joint prosthesis according to claim 11, wherein the step size between the individual displacement steps is in the range from about 0.5 mm to 2.0 mm.

14. Shoulder joint prosthesis, especially according to claim 1, there is provided a set of joint parts, in the case of a reverse shoulder joint prosthesis a set of humeral sockets, the joint surfaces of which have angles of inclination of differing magnitudes relative to the shaft or to the longitudinal mid-axis of the latter.

15. Shoulder joint prosthesis according to claim 1, the joint part associated with the humeral shaft, in the case of a reverse shoulder joint prosthesis the humeral socket, is held so as to be tiltable relative to the shaft, at least in the antero-posterior direction, especially in the manner of a universal joint.

16. Shoulder joint prosthesis, especially according to claim 1, wherein the joint part on the humerus side, in the case of a reverse shoulder joint prosthesis the humeral socket, includes an inlay which is separately insertable, especially arranged to be latched into place, which is made of polyethylene, ceramics, stainless steel or like bearing material tolerated by humans and which defines the joint surface of this joint part.

17. A reverse shoulder joint prosthesis having a joint part, connectible at the proximal end of a humeral shaft, wherein the joint part associated with the humeral shaft, in the case of a reverse shoulder joint prosthesis the humeral socket, is held displaceably or displaced relative to the shaft at least linearly.

18. Shoulder joint prosthesis according to claim 12, wherein the maximum displacement in the postero-anterior direction and/or in the medio lateral direction and/or in the proximal-distal direction is in the range between 4.0 mm and 8.0 mm.

19. Shoulder joint prosthesis according to claim 13, wherein the step size between the individual displacement steps is in the range from about 1.0 mm to 1.5 mm.
